(19) United States
(12) Patent Application Publication
(10) Pub. No.: US 2022/0047407 A1
(43) Pub. Date: Feb. 17, 2022

(54) Open-Fingered Hand Support Brace

(71) Applicant: Rebecca A. Baril, South Lyon, MI (US)

(72) Inventor: Rebecca A. Baril, South Lyon, MI (US)

(21) Appl. No.: 16/991,926

(22) Filed: Aug. 12, 2020

(51) Int. Cl. A61F 5/01; A61F 5/30

(52) U.S. Cl. CPC A61F 5/0118; A61F 5/30

(57) ABSTRACT

An open-fingered hand support brace is an apparatus that supports a slight rectus position for the fingers of a hand, while eliminating contractures from daily activities and deformities or conditions that may lead to contractures and pain. The apparatus includes a support sleeve and a support cushion. The support sleeve wraps around fingers of a hand and preferably applies slight compression. The support cushion orients the fingers of the hand with respect to the palm. The support sleeve includes a fingers-bracing portion, a palm-bracing portion, and a wrist-bracing portion in order to protect the hand. The support sleeve further includes a wrist-receiving rim in order for a hand to freely traverse into and out of the support sleeve. The support sleeve further includes a fingers-receiving rim and a thumb-receiving rim in order for the fingers and the thumb of a hand to be exposed while braced with the support sleeve.

### FIELD OF THE INVENTION

The present invention generally relates to hand braces. More specifically, the present invention is an open-fingered hand support brace.

### BACKGROUND OF THE INVENTION

Society of all ages have stress on their fingers all day, from writing, typing, clenching the steering wheel and all types of careers cause contractures of the fingers. Even young children learning to write and playing on gaming devices contract their fingers practically all day long. Regardless of the severity of stress for a hand, a hand needs support to relieve contractures of fingers from daily activities. Existing braces are uncomfortable, bulky, and can even further limit the abilities of a hand. A hand is suffocated within the brace leaving marks of the brace on the hand and does not allow the skin to breathe. Both the range of motion for the fingers and the wrist are extremely limited and strain the hand even further throughout prolonged use.

It is therefore an objective of the present invention to provide a comfortable brace for fingers that are sore and swollen from daily use of a user. The user may be an individual of any age. The present invention that allows for more range of motion while preventing the fingers from being sore, swollen, and contracted all day from typical daily activities and even sleeping. The fingers are in a contracted position to some degree all day 24 hours and never extend unless you control the fingers to extend and you cannot hold this extended position for long periods of time without the engagement of the present invention. The present invention may also be used to relieve stress on the fingers and also can be used to help with hand injuries. The present invention is easily secured with and removed from a hand. The present invention, more specifically, allows the fingers to freely and comfortably move. As the support of the present invention is separable from the brace, the present invention is easily cleaned and maintained. The present invention further accommodates more users than that of existing braces as the present invention stretches to conform around a hand. The support is removable and therefore interchangeable according to the needs of the hand.

### BRIEF DESCRIPTION OF THE DRAWINGS

FIG. 1 is a perspective view of a support sleeve of the present invention.

FIG. 2 is a perspective view of a support cushion being engaged into a support sleeve of the present invention with a fingers-bracing portion being stretched over the support cushion.

FIG. 3 is a perspective view of the support cushion being engaged within the support sleeve of the present invention.

FIG. 4 is a perspective view of a fingers cap with the support sleeve of the present invention in a storage configuration.

FIG. 5 is a perspective view of the fingers cap with the support sleeve of the present invention in an operative configuration.

### DETAIL DESCRIPTIONS OF THE INVENTION

All illustrations of the drawings are for the purpose of describing selected versions of the present invention and are not intended to limit the scope of the present invention.

The present invention is an open-fingered hand support brace. The present invention allows the sore contracted fingers of a hand to move freely while engaged with the present invention. The present invention is easily worn and removed by a user. The present invention defines the structure of the hand. The present invention serves as a preventative method to relieve contractures of fingers from daily activities, such as typing, driving, playing video-games, cooking and other activities that make you grasp your fingers, tightening fingers, clamping, gripping, flexion of digits of the hand and making a fist motion. The present invention is used to alleviate tired finger and soreness after doing regular daily activities from brushing your hair to playing golf, opening jars for cooking. Typical daily routines place enormous stress on your fingers and constant clasping of your fingers in a fist like motion. The present invention is preferably worn on a daily basis to rest the fingers and stretch them out. At night, while sleeping, a user may make a fist which adds additional strain on the fingers from daily activities. The present invention is preferably worn for users with clenched hands while sleeping in order to prevent tightening of the fingers. The present invention is ideally worn while sleeping to prevent tight clenching of the fingertips. Clenching of the fingertips impedes blood flow to the fingers and causes swelling and pain in the fingers when one awakens. The present invention may also be worn during relaxing periods of time to straighten fingers and relieve strain. The present invention also alleviates minor and serious injuries, such a tendonitis, dislocations, neuropathies and arthritis. Moreover, the present invention helps elongate the fingers and prevents contracting the fingers.

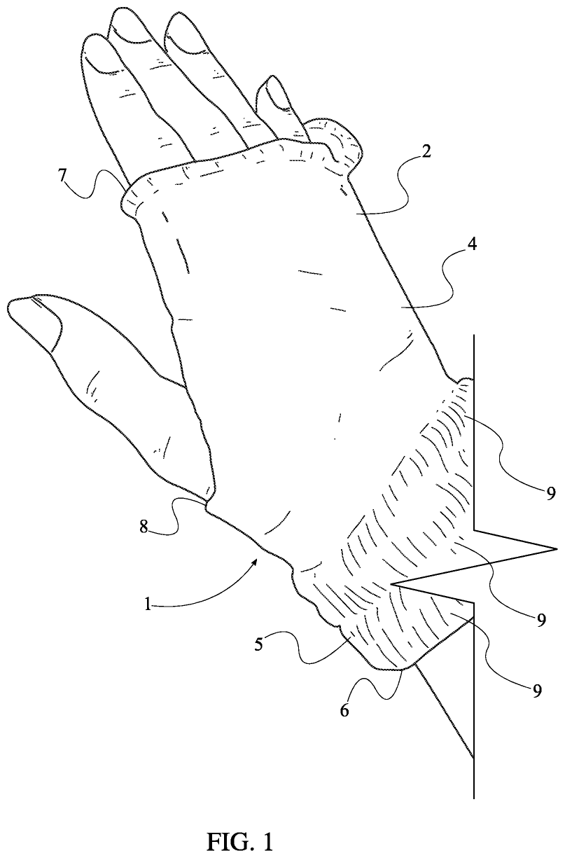

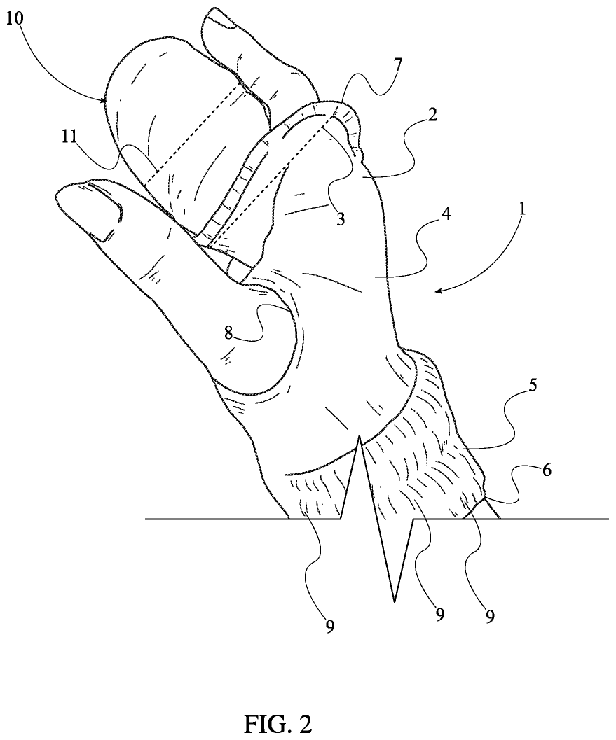

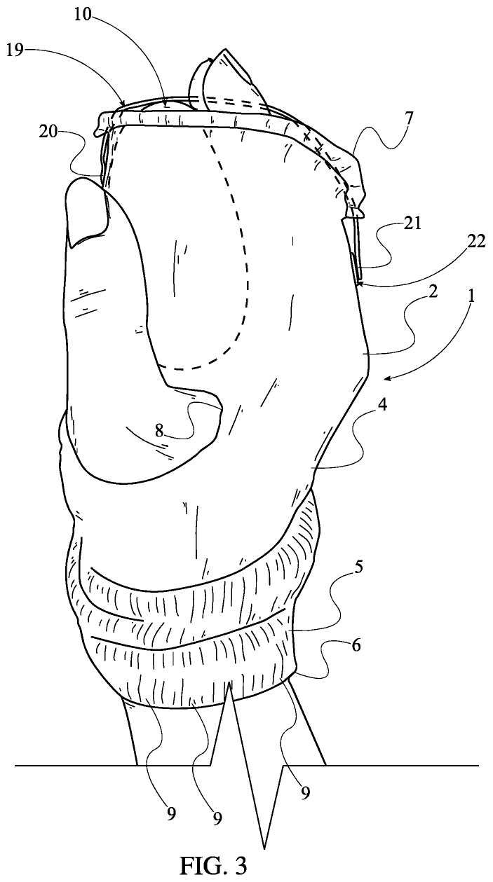

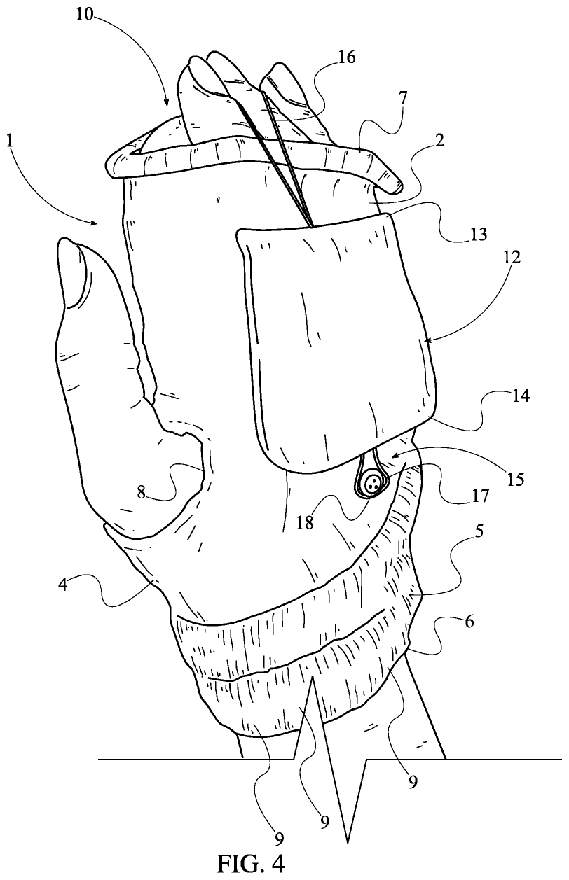

In order for the present invention to properly support the hand and elongate the fingers, a preferred embodiment of the present invention comprises a support sleeve 1 and a support cushion 10, seen in FIG. 2, FIG. 3, and FIG. 4. As seen in FIG. 1, the support sleeve 1 surrounds the hand and secures the support cushion 10 against the hand. The support sleeve 1 preferably applies slight compression as the support sleeve 1 is made of elastic fabric material. The support cushion 10 is relatively firm and provides contours that allows the hand to comfortably conform the hand into a desired position. The desired position of the support cushion 10 is along the palm surface and extends to the fingers, allowing the injuries, tight contractures, and pain in the finger joints of the hand to properly heal. More specifically, the support cushion 10 extends from the proximal ends of the fingers to the interphalangeal joint of each finger. The support cushion 10 remains pressed against the palmar surface of the hand of the user and the support sleeve 1 supports the hand as well as the support sleeve 1 comprises a fingers-bracing portion 2, a palm-bracing portion 4, a wrist-bracing portion 5, a wrist-receiving rim 6, a fingers-receiving rim 7, and a thumb-receiving rim 8. The fingers-bracing portion 2 surrounds the fingers of the hand, the palm-bracing portion 4 surrounds the palm of the hand, and the wrist-bracing portion 5 surrounds the wrist of the hand. The wrist-receiving rim 6 allows the hand to enter the support sleeve 1 and secures the wrist-bracing portion 5 around the wrist. The fingers-receiving rim 7 allows the fingers of the hand to traverse through the support sleeve 1 and secures the fingers-bracing portion 2 around the fingers of the hand. The thumb-receiving rim 8 allows the thumb of the hand to traverse through the support sleeve 1 and secures the palm-bracing portion 4 around palm of the hand.

The overall configuration of the aforementioned components allows the support cushion 10 to effectively position and support the fingers of a hand in a straightened rectus position while preserving the comfort of the hand, seen in FIG. 1, FIG. 2, FIG. 3, and FIG. 4. In order for the support sleeve 1 to press against the contours of the hand, the fingers-bracing portion 2 is positioned adjacent with the palm-bracing portion 4, and the wrist-bracing portion 5 is positioned adjacent with the palm-bracing portion 4, opposite with the fingers-bracing portion 2. This arrangement defines an ergonomic structure for the support sleeve 1. Furthermore, the fingers-receiving rim 7 is positioned adjacent with the fingers-bracing portion 2, opposite the palm-bracing portion 4. Likewise, the wrist-receiving rim 6 is positioned adjacent with the wrist-receiving portion 5, opposite the palm-bracing portion 4. This arrangement allows the hand to enter and exit the support sleeve 1 continuously and freely. Moreover, the thumb-receiving rim 8 is laterally positioned with the palm-bracing portion 4 and is oriented perpendicular with the fingers-receiving rim 7 to accommodate the position of the thumb with respect to the rest of the hand. Moreover, the thumb-receiving rim 8 allows the thumb to traverse through the palm-bracing portion 4 and move freely. The support cushion 10 is situated within the support sleeve 1 in order to position the hand for optimal recovery throughout daily movement. More specifically, the support cushion 10 is pressed against the fingers-bracing portion 2 for a natural curved form and more rectus straightened position of the finger joints.

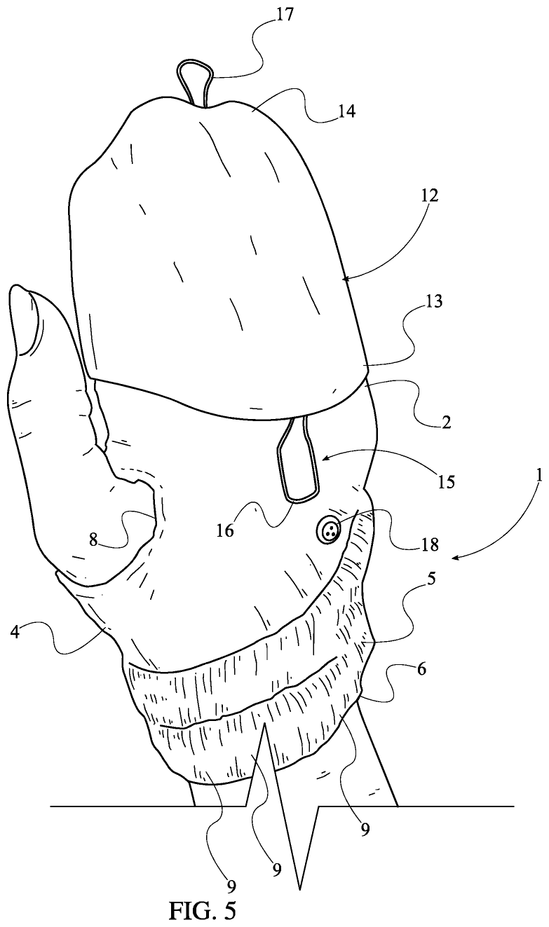

In order to properly support the wrist of a hand, the support sleeve 1 may further comprise a plurality of elastic bands 9, seen in FIG. 1, FIG. 2, FIG. 3, FIG. 4, and FIG. 5. The plurality of elastic bands 9 tightens and gently grips around the wrist of the user to gently guide and support the wrist throughout daily movements. As the wrist of the hand is surrounded by the wrist-bracing portion 5, the plurality of elastic bands 9 is distributed along the wrist-bracing portion 5. More specifically, each of the plurality of elastic bands 9 is integrated into and around the wrist-bracing portion 5.

The comfort of the fingers for a hand is further preserved as the present invention may further comprise a fingers cap 12, seen in FIG. 4 and FIG. 5. The fingers cap 12 encloses the exposed portions of the fingers that traverse through the fingers-bracing portion 2. The fingers cap 12 provides warmth and comfort for the exposed portions of the fingers. The fingers of the hand may be enclosed or freed with the fingers cap 12 as the fingers cap 12 is operatively coupled to the fingers-receiving rim 7, wherein the fingers cap 12 is used to expose and cover the fingers-receiving rim 7. Wherein the support sleeve 1 and the fingers cap 12 are arranged into an operative configuration, seen in FIG. 4, the fingers cap 12 encloses the exposed the portions of the fingers with the fingers-bracing portion 2. The fingers cap 12 comprises an open end 13 and a closed end 14. The fingers enter through the open end 13 and are shielded by the closed end 14. The open end 13 is positioned opposite the closed end 14 about the fingers cap 12, accommodating the linear structure of the fingers. The fingers are completely shielded with the fingers cap 12 and the support sleeve 1 as the open end 13 is positioned around the fingers-receiving rim 7. More specifically, the closed end 14 is positioned offset from the fingers-receiving rim 7.

Likewise, wherein the support sleeve 1, the fingers cap 12, and a cap fastener 15 are arranged into a storage configuration, seen in FIG. 5, the fingers cap 12 remains attached with the support sleeve 1 and a hand when not in use. The cap fastener 15 comprises a first loop 16, a second loop 17, and a rigid member 18. The first loop 16 latches the fingers cap 12 with a finger of the hand, preferably the middle finger for stability, and the second loop 17 latches the fingers cap 12 with the rigid member 18. The rigid member 18 connects the second loop 17 with the support sleeve 1. The rigid member 18 is preferably a button. Similarly, with the storage configuration, the fingers cap 12 comprises an open end 13 and a closed end 14, and the open end 13 is positioned opposite the closed end 14 about the fingers cap 12. In order for the fingers cap 12 to remain taut with the support sleeve 1 will in the storage configuration, the first loop 16 is fixed adjacent with the open end 13 and is oriented towards the fingers-receiving rim 7. Similarly, the second loop 17 is fixed adjacent with the closed end 14. The rigid member 18 is externally mounted with the palm-bracing portion 4 so that the second loop 17 may be easily connected and disconnected with the rigid member 18. The fingers cap 12 remains stretched across and connected with the present invention as the first loop 16 is engaged by the middle finger of the hand and the second loop 17 is engaged by the rigid member 18.

In order for the support brace to position and support the fingers of a hand, the support cushion 10 is a spherical shape, seen in FIG. 2 and FIG. 3. The spherical shape contours the fingers, allowing some degree of motion and curve, while simultaneously allowing straightening and extending of the finger joints with the support cushion 10 so that the fingers are effectively positioned for a full recovery. More specifically, a diameter 11 of the support cushion 10 is greater than or equal to a radius 3 of a transversal cross section of the fingers-bracing portion 2. In this preferred embodiment of the present invention the diameter 11 further compresses and reinforces the desired position of the hand within the support sleeve 1.

The support cushion 10 is further secured within the support sleeve 1 while allowing the fingers of a hand to remain exposed as the present invention may further comprise a restraining strap 19 and a strap fastener 22, seen in FIG. 3. The restraining strap 22 stops the support cushion 10 from exiting the support sleeve 1 and exiting past the fingers-receiving rim 7. Moreover, the restraining strap 19 is preferably flat and is positioned in between two adjacent fingers while secured with the support sleeve 1. The strap fastener 22 secures and releases the restraining strap 19 with the support sleeve 1. The strap fastener 22 is preferably a couple of hook-and-loop fastening patches that engage with one another. The restraining strap 19 comprises a first strap end 20 and a second strap end 21. The first strap end 20 fastens the restraining strap 19 with the support sleeve 1, and the second strap end 21 secures the restraining strap 19 across the fingers-receiving rim 7. In order for the support cushion 10 to be stopped with the restraining strap 19, the restraining strap 19 is positioned across the fingers-receiving rim 7. The first strap end 20 is laterally connected to the fingers-bracing portion 2, so that the restraining strap 19 easily reaches over and across the fingers-bracing portion 2. The position of the restraining strap 19 is secured over and across the fingers-bracing portion 2 as second strap end 21 is laterally attached to the fingers-bracing portion 2 by the strap fastener 22, opposite the first strap end 20.

Although the invention has been explained in relation to its preferred embodiment, it is to be understood that many other possible modifications and variations can be made without departing from the spirit and scope of the invention as hereinafter claimed.

What is claimed is:

1. An open-fingered hand support brace comprises:
a support sleeve;
a support cushion;
the support sleeve comprises a fingers-bracing portion, a palm-bracing portion, a wrist-bracing portion, a wrist-receiving rim, a fingers-receiving rim, and a thumb-receiving rim;
the fingers-bracing portion being positioned adjacent with the palm-bracing portion;
the wrist-bracing portion being positioned adjacent with the palm-bracing portion, opposite with the fingers-bracing portion;
the fingers-receiving rim being positioned adjacent with the fingers-bracing portion, opposite the palm-bracing portion;
the wrist-receiving rim being positioned adjacent with the wrist-receiving portion, opposite the palm-bracing portion;
the thumb-receiving rim being laterally positioned with the palm-bracing portion;
the thumb-receiving rim being oriented perpendicular with the fingers-receiving rim; and,
the support cushion being situated within the support sleeve.

2. The open-fingered hand support brace as claimed in claim 1 comprises:
the support cushion being pressed against the fingers-bracing portion.

3. The open-fingered hand support brace as claimed in claim 1, wherein the support sleeve is made of an elastic fabric material.

4. The open-fingered hand support brace as claimed in claim 1 comprises:
the support sleeve further comprises a plurality of elastic bands;
the plurality of elastic bands being distributed along the wrist-bracing portion; and,
each of the plurality of elastic bands being integrated into and around the wrist-bracing portion.

5. The open-fingered hand support brace as claimed in claim 1 comprises:
a fingers cap; and,
the fingers cap being operatively coupled to the fingers-receiving rim, wherein the fingers cap is used to expose and cover the fingers-receiving rim.

6. The open-fingered hand support brace as claimed in claim 5 comprises:
wherein the support sleeve and the fingers cap are arranged into an operative configuration;
the fingers cap comprises an open end and a closed end;
the open end being positioned opposite the closed end about the fingers cap;
the open end being positioned around the fingers-receiving rim; and,
the closed end being positioned offset from the fingers-receiving rim.

7. The open-fingered hand support brace as claimed in claim 5 comprises:
wherein the support sleeve, the fingers cap, and a cap fastener are arranged into a storage configuration;
the cap fastener comprises a first loop, a second loop, and a rigid member;
the fingers cap comprises an open end and a closed end;
the open end being positioned opposite the closed end about the fingers cap;
the first loop being fixed adjacent with the open end;
the first loop being oriented towards the fingers-receiving rim;
the second loop being fixed adjacent with the closed end;
the rigid member being externally mounted with the palm-bracing portion; and,
the second loop being engaged by the rigid member.

8. The open-fingered hand support brace as claimed in claim 1, wherein the support cushion is a spherical shape.

9. The open-fingered hand support brace as claimed in claim 8, wherein a diameter of the support cushion is greater than or equal to a radius of a transversal cross section of the fingers-bracing portion.

10. The open-fingered hand support brace as claimed in claim 1 comprises:
a restraining strap;
a strap fastener;
the restraining strap comprises a first strap end and a second strap end;
the restraining strap being positioned across the fingers-receiving rim;
the first strap end being laterally connected to the fingers-bracing portion; and,
the second strap end being laterally attached to the fingers-bracing portion by the strap fastener, opposite the first strap end.

11. An open-fingered hand support brace comprises:
a support sleeve;
a support cushion;
a fingers cap;
the support sleeve comprises a fingers-bracing portion, a palm-bracing portion, a wrist-bracing portion, a wrist-receiving rim, a fingers-receiving rim, and a thumb-receiving rim;
the fingers-bracing portion being positioned adjacent with the palm-bracing portion;
the wrist-bracing portion being positioned adjacent with the palm-bracing portion, opposite with the fingers-bracing portion;
the fingers-receiving rim being positioned adjacent with the fingers-bracing portion, opposite the palm-bracing portion;
the wrist-receiving rim being positioned adjacent with the wrist-receiving portion, opposite the palm-bracing portion;
the thumb-receiving rim being laterally positioned with the palm-bracing portion;
the thumb-receiving rim being oriented perpendicular with the fingers-receiving rim;
the support cushion being situated within the support sleeve; and, the fingers cap being operatively coupled to the fingers-receiving rim, wherein the fingers cap is used to expose and cover the fingers-receiving rim.

12. The open-fingered hand support brace as claimed in claim 11 comprises:
the support cushion being pressed against the fingers-bracing portion.

13. The open-fingered hand support brace as claimed in claim 11, wherein the support sleeve is made of an elastic fabric material.

14. The open-fingered hand support brace as claimed in claim 11 comprises:
the support sleeve further comprises a plurality of elastic bands;
the plurality of elastic bands being distributed along the wrist-bracing portion; and,
each of the plurality of elastic bands being integrated into and around the wrist-bracing portion.

15. The open-fingered hand support brace as claimed in claim 11 comprises:
wherein the support sleeve and the fingers cap are arranged into an operative configuration;
the fingers cap comprises an open end and a closed end;
the open end being positioned opposite the closed end about the fingers cap;
the open end being positioned around the fingers-receiving rim; and,
the closed end being positioned offset from the fingers-receiving rim.

16. The open-fingered hand support brace as claimed in claim 11 comprises:
wherein the support sleeve, the fingers cap, and a cap fastener are arranged into a storage configuration;
the cap fastener comprises a first loop, a second loop, and a rigid member;
the fingers cap comprises an open end and a closed end;
the open end being positioned opposite the closed end about the fingers cap;
the first loop being fixed adjacent with the open end;
the first loop being oriented towards the fingers-receiving rim;
the second loop being fixed adjacent with the closed end;
the rigid member being externally mounted with the palm-bracing portion; and,
the second loop being engaged by the rigid member.

17. The open-fingered hand support brace as claimed in claim 11, wherein the support cushion is a spherical shape, and wherein a diameter of the support cushion is greater than or equal to a radius of a transversal cross section of the fingers-bracing portion.

18. The open-fingered hand support brace as claimed in claim 11 comprises:
a restraining strap;
a strap fastener;
the restraining strap comprises a first strap end and a second strap end;
the restraining strap being positioned across the fingers-receiving rim;
the first strap end being laterally connected to the fingers-bracing portion; and,
the second strap end being laterally attached to the fingers-bracing portion by the strap fastener, opposite the first strap end.
